(19) United States
(12) Patent Application Publication
(10) Pub. No.: US 2011/0065830 A1
(43) Pub. Date: Mar. 17, 2011

(54) Hydrogel Denture Adhesive

(76) Inventor: Michael J. Borja, Keyport, NJ (US)

(21) Appl. No.: 12/558,125

(22) Filed: Sep. 11, 2009

(51) Int. Cl. A61K 6/00

(52) U.S. Cl. 523/120

(57) ABSTRACT

A hydrogel denture adhesive comprising an AVE/MA Salt in an amount of about 20 to about 80 percent by weight of the hydrogel, a suitable humectant in an amount of about 1 to about 30 percent by weight of the hydrogel, and water in an amount of about 20 to about 80 percent by weight of the hydrogel.

### FIELD OF THE INVENTION

This invention relates to dental compositions based on blends of particular constituents and to the process of making such compositions, in particular to formulations and processes for making denture adhesives.

### BACKGROUND OF THE INVENTION

Denture adhesives are used to assist a denture's ability to adhere to the gums. Denture adhesives are typically characterized as contributing an adhesive function once exposed to the moisture in a person's mouth and dissolve in the mouth over the course of a day.

A conventional approach is to provide an adhesive material made of a resin in the form of a powder or paste, which can be either natural or synthetic. Each time a user needs to apply a denture to the gum, the user takes the required amount of a denture base stabilizing material from a tube in the case of a powder or paste. Conventional denture adhesives in the form of pastes and powders are highly hydrophilic and readily absorb moisture forming an adhesive gel. One example is conventional Gantrez-based denture adhesives, which are applied to the denture and activated by saliva when the denture is first inserted into the mouth. These pastes and powders can be problematic for denture users. Once inserted, force is applied to the denture in order to spread the paste or power and ensure a snug fit of the denture to the gums. The force of application will cause the gums to displace the paste or gel that is formed and these adhesives will typically ooze or squirt out the side of the denture. This is known as primary ooze and is considered by many denture users as objectionable. Pastes and powders can be difficult to use because they require the denture wearer to ensure that the paste or powder is uniformly applied and in the right amount. Similar and other types of soft denture liners or adhesives have been disclosed in Graham et al, J., Prosthetic Dentistry, Volume 2, No. 4, pp. 422-8 (1989) and U.S. Pat. Nos. 5,061,182, 5,075,107, 5,436,283, and 5,513,988.

One approach for solving the problems of conventional denture adhesives is to provide a denture liner with mucoadhesive properties. U.S. Pat. No. 7,312,256 attempts to resolve known deficiencies in the art by providing a denture liner with a hydrophobic polymer component, such as polyethyl methacrylate, an esterified copolymer of methyl vinyl ether and maleic anhydride, polyvinyl acetate that inhibits the liner from absorbing moisture, a plasticizing component such as glycerol triacetate, an adhesive component such as sodium carboxymethylcellulose or polyethylene oxide. Another approach, disclosed in U.S. Pat. Nos. 6,583,225, 6,706,817, and 7,288,597, is to form a hydrogel through the cross-linking reaction between an acidic polymer, particularly copolymers of maleic anhydride or maleic acid, and a comonomer, e.g. an alkylvinylether, with a suitable crosslinking agent, such as glycerol, in the presence of an esterification or amidization catalyst, such as sulfuric acid. The disclosed hydrogel is a covalently bound, ester and/or amide/imide crosslinked hydrogel that is extremely swellable in the presence of moisture. This hydrogel would be tacky in the unswelled state, but tackiness diminishes as swelling increases. Such a product would not function effectively as a denture adhesive because any significant swelling upon exposure to saliva would adversely affect adhesion and bite. As a result, an additional layer or component of a conventional denture adhesive would likely need to be included with the hydrogel to ensure proper adhesion of the denture to the gums.

It is an object of the present invention to provide a hydrogel with improved adhesive properties to serve as a denture adhesive. It is a further object that such a hydrogel have an immediate or quick onset of adhesive action and avoids the deficiencies associated with primary ooze. It is still a further object of the present invention to provide a hydrogel denture adhesive that is resistant to dissolving over an extended period of time in the presence of water so as to provide an adhesive with an extended lifetime for effective use.

### SUMMARY OF THE INVENTION

These and other objects are achieved by providing a hydrogel denture adhesive with an AVE/MA Salt in an amount of about 20 to about 80 percent by weight of the hydrogel, a suitable humectant in an amount of about 1 to about 30 percent by weight of the hydrogel, and water in an amount of about 20 to about 80 percent by weight of the hydrogel. The AVE/MA Salt is a salt of poly (lower alkyl vinyl ether/maleic anhydride). The AVE/MA Salt may also be provided in an amount of about 30 to about 60 percent by weight of the hydrogel or in an amount of about 50 percent by weight of the hydrogel. Water may also be provided in an amount of about 30 to about 60 percent by weight of the hydrogel or in an amount of about 35 to about 45 percent by weight of the hydrogel. The suitable humectant can be selected from a group consisting of glycerin, polyhydric alcohols, ethylene glycol, propylene glycol, polyethylene glycol, sorbitol, and combinations thereof. The suitable humectant may also be provided in an amount of about 5 to about 20 percent by weight of the hydrogel or in an amount of about 8 to about 12 percent by weight of the hydrogel.

The hydrogel denture adhesive of the present invention can also include a suitable rheological agent in an amount of about 0.1 to about 10 percent by weight of the hydrogel or in an amount of about 0.1 to about 5 percent by weight of the hydrogel.

The hydrogel denture adhesive of the present invention can further include an additional ingredient selected from a group consisting of an antimicrobial agent, an antibiotic, an anti-inflammatory agent, a dental desensitizing agent, an anesthetic agent, an anti-fungal, an aromatic, insulin, a steroid, an anti-neoplastic, a colorant, a preservative, a flavor component, a fragrance component, a sensation component, and combinations thereof.

The objects of the present invention can also be achieved by providing a hydrogel denture adhesive prepared by a process comprising the steps of: a) mixing a suitable humectant in an amount of about 1 to about 30 percent by weight of the hydrogel with water in an amount of about 20 to about 80 percent by weight of the hydrogel; b) heating the mixture and c) dispersing an AVE/MA Salt in an amount of about 20 to about 80 percent by weight of the hydrogel in the mixture. The process can further include the step of heating the suitable humectant to 50-80° C. prior to the step of mixing the suitable humectant with water. The step of cooling the dispersion can further comprise the step of cooling the dispersion or cooling the mixture prior to the step of dispersing the AVE/MA Salt.

The process can still further include the steps of mixing an AVE/MA Copolymer with a cross linking agent and a neutralizing agent, heating the mixture in solution to a cross-linking temperature and forming the AVE/MA Salt, and cooling the AVE/MA Salt. The cross-linking agent can be a multivalent cation and the neutralizing agent is a monovalent cation.

Other objects of the invention and its particular features and advantages will become more apparent from consideration of the accompanying description.

### DETAILED DESCRIPTION OF THE INVENTION

A denture adhesive of the present invention is formed by a mixture of an AVE/MA Salt component, a suitable humectant and water in effective proportions to form a hydrogel denture adhesive with improved adhesive characteristics.

As noted above, the AVE/MA Salt is a salt of poly (lower alkyl vinyl ether/maleic anhydride), including the salts of poly (lower alkyl vinyl ether/maleic anhydride) disclosed in U.S. Pat. Nos. 4,373,036, 4,758,630, 4,980,391, 5,037,924, 5,073,604, 5,093,387, 5,298,534, and 5,525,652, which are incorporated herein by reference as if fully set forth herein. Preferred AVE/MA Salts include Ca/Na salts of poly (lower alkyl vinyl ether/maleic anhydride) and are provided by the ISP Corporation of Wayne, N.J. under the tradename “Gantrez® MS-955.” Gantrez® MS-955 is a mixed sodium and calcium salt of poly (methylvinyl ether/maleic anhydride) and is described by the chemical structural formula:

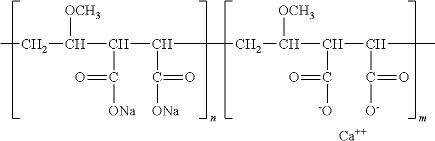

where n is an integer of 1 or greater, and m is an integer of 1 or greater.

The AVE/MA Salt is a hydrophilic polymer that provides the adhesive characteristics of the hydrogel denture adhesive. The AVE/MA Salt component can be in an amount of about 20 to about 80 percent by weight of the hydrogel, preferably in an amount of about 30 to about 60 percent by weight of the hydrogel, most preferably in an amount of about 45 to about 55 percent by weight of the hydrogel.

The suitable humectant is used to provide a moistening effect to the hydrogel, to hydrate the AVE/MA Salt, and provide stability to the hydrogel over an extended period of time. The suitable humectant provides stability by limiting or controlling the rate at which the hydrogel denture adhesive absorbs water over time when placed in the mouth and thus controls the rate at which the AVE/MA Salt hydrates and dissolves in the mouth. Suitable humectants include, but are not limited to, glycerin, polyhydric alcohols such as ethylene glycol, propylene glycol, polyethylene glycol, and sorbitol, and combinations thereof. The suitable humectant can be in an amount of about 1 to about 30 percent by weight of the composition, preferably in an amount of about 5 to about 20 percent by weight of the hydrogel, most preferably in an amount of about 8 to about 12 percent by weight of the hydrogel. The preferred suitable humectant is glycerin.

The third component of the hydrogel is water, which can be present in an amount of about 20 to about 80 percent by weight of the hydrogel, preferably in an amount of about 30 to about 60 percent by weight of the hydrogel, most preferably in an amount of about 35 to about 45 percent by weight of the hydrogel. By combining water with the AVE/MA Salt, the adhesive characteristics of the hydrogel are already activated and the hydrogel is able to adhere a denture to the gums immediately upon application. This is a characteristic that is not shared by other known adhesives. In addition to providing body to the hydrogel denture adhesive, incorporating a significant proportion of water enables the hydrogel to provide a cushion between a person's gums and the denture, improving overall comfort.

An optional fourth component of the hydrogel is a suitable rheological agent that will assist the hydrogel in resisting deformation. Suitable rheological agents include cellulosic polymers such as sodium CMC, HPMC, HEMC, sodium alginate, karaya gum, xanthan gum, fumed silica (Si0₂) and silicate clays. The suitable rheological agent can be in an amount of about 0.1 to about 10 percent by weight of the composition, preferably in an amount of about 0.1 to about 5 percent by weight of the hydrogel. The preferred suitable rheological agent is fumed silica.

The denture adhesive according to the present invention can contain additional ingredients. These ingredients can improve the functionality of the adhesive and/or provide ancillary benefits to the denture wearer.

Additional ingredients, which can be used in the denture adhesive in accordance with the present invention, include therapeutically active agents suitable for treating individuals in need thereof. These agents include antimicrobial agents, such as iodine, sulfonamides, bisbiguanides, or phenolics, antibiotics such as tetracycline, neomycin, kanamycin, metronidazole, or clindamycin, anti-inflammatory agents, such as aspirin, acetaminophen, naproxen and its salts, ibuprofen, ketorolac, flurbiprofen, indomethacin, eugenol, or hydrocortisone, dental desensitizing agents, such as potassium nitrate, strontium chloride or sodium fluoride, anesthetic agents, such as lidocaine or benzocaine; anti-fungals, aromatics, such as camphor, eucalyptus oil, and aldehyde derivatives, such as benzaldehyde; insulin, steroids, and anti-neoplastics. Certain forms of therapy and combinations of these agents in the same delivery system may be useful in order to obtain an optimal effect.

Other suitable additional ingredients include colorants and preservatives, such as methyl and propyl parabens. The denture adhesive compositions of the present invention may also include one or more components, which provide flavor, fragrance, and/or sensation benefit. These components include natural or artificial sweetening agents, menthol, menthyl lactate, wintergreen oil, peppermint oil, spearmint oil, leaf alcohol, as well as coolants 3-1-menthoxypropane-1,2-diol and paramenthane carboxyamide agents, such as N-ethyl-p-menthane-3-carboxamide.

When the components of the present invention are combined in effective proportions, a hydrogel is formed with properties that are particularly beneficial for use as a denture adhesive. The use of a suitable humectant, such as glycerin, is beneficial because the combination of this component with an AVE/MA Salt component and water in proper proportions will produce an adhesive hydrogel of optimum solubility that limits the degree of swelling of the overall hydrogel and resists dissolution in the presence of mouth moisture. A suitable humectant scavenges water from the AVE/MA Salt component and acts as a hydrophilic suspending agent. As a result, the suitable humectant controls the amount of water taken up by the AVE/MA salt, so that the end product has the desired amount of gelling, hardness/softness and a slow hydration rate. The suitable humectant also serves as a vehicle that forms weak hydrogen bonds with the free acid groups in the AVE/MA salt which also helps to achieve the desired amount of gelling. If a suitable humectant is not used in proper proportions, the hydrogel possesses undesirable properties. Too much of a suitable humectant will not allow the AVE/MA Salt component to solubilize, and too little will produce a hydrogel that is too tacky and unmanageable for use as a denture adhesive. The use of a suitable humectant, such as glycerin, is also beneficial because this component can also provide therapeutic benefits of a warming sensation and lubricity in making the mouth feel better due to its emollient properties.

The following are non-limiting examples of denture adhesives in accordance with the present invention.

### EXAMPLES

| | Example 1 | Example 2 | Example 3 | Example 4 |
|---|---|---|---|---|
| | (weight | (weight | (weight | (weight |
| Ingredient | %) | %) | %) | %) |
| DI Water | 34.94 | 39.94 | 34.94 | 34.94 |
| Glycerin | 10.0 | 10.0 | 15.0 | 10.0 |
| Gantrez MS955 | 55.0 | 50.0 | 50.0 | 54.99-50.0 |
| Cetylpyridinium | 10 0.05 | 0.05 | 0.05 | 0.05 |
| Chloride | | | | |
| D&C Red No. 27 | 0.01 | 0.01 | 0.01 | 0.01 |
| Aluminum Lake | | | | |
| Cab-O-Sil M5 SiO2 | — | — | — | 0.01-5.0 |
| (silicon dioxide) | | | | |

The Gantrez MS955 and other AVE/MA Salts for use in the present invention can be made from a copolymer of methyl vinyl ether and maleic anhydride (hereinafter “AVE/MA Copolymer”) described by the chemical structural formula:

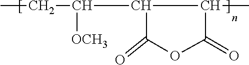

where n is an integer of 1 or greater.
   Such copolymers are commercially available under the trade name Gantrez AN and include AN-119, AN-903, AN-139, and AN-169. The AVE/MA Salts can be made by mixing the AVE/MA Copolymer with a multivalent cation that serves as a cross linking agent and a monovalent cation that serves as a neutralizing agent. The multivalent cations are preferably alkali metal cations, most preferably magnesium and calcium. The monovalent cations are also preferably alkali metal cations, most preferably sodium and potassium cations. The mixture of the AVE/MA Copolymer, multivalent cations and monovalent cations are placed in solution and the solution is heated to a copolymer cross-linking temperature, which can range from 85° C. to 110° C., forming the AVE/MA salt. After formation of the AVE/MA salt, the composition is cooled and any remaining water is removed. One example for forming the AVE/MA Salt is in accordance with the following method: 1) dry mix 76.3 w/w % Gantrez AN-169 BF with 21.7 w/w % Ca(OH)2 and 2.0 w/w % NaOH; 2) combine the dry mixture with DI water in a reactor vessel to achieve a 5-15% solution/slurry; 3) heat the solution/slurry to 85° C. and mix for an hour; 4) transfer the solution/slurry to a drying tray and flash off water via evaporation using an oven at 65° C.; and 5) cool and mill the material into a fine powder. Other examples for forming the AVE/MA Salt are disclosed in U.S. Pat. Nos. 4,373,036, 4,758,630, 4,980,391, 5,037,924, 5,073,604, 5,093,387, 5,298,534, and 5,525,652, which are incorporated herein by reference as if fully set forth herein.

The hydrogel of the above examples may be made by charging the requisite amount of glycerin, which can be 10 or 15 grams, into a suitable container and heating to 65° C. 0.01 grams of D&C Red No. 27 Aluminum Lake and 0.05 grams of Cetylpyridinium Chloride are added to the container and the combination is mixed until the mixture is homogenous. The requisite amount of water, which can be 34.94 or 39.94 grams, is added to the container and the mixture is mixed for 5 minutes. The requisite amount of Gantrez MS955, which can be 55 or 50 grams, is added to the container and mixed until the Gantrez MS955 is dispersed. Once the Gantrez MS955 is dispersed, the mixture is poured immediately on to a cooling tray and cooled to room temperature. An alternative to this process is to cool the mixture of water and glycerin to room temperature or below and subsequently add Gantrez MS955 until the polymer is dispersed.

The hydrogel of example 4 can be made primarily following the preceding processes, however the processes are slightly modified to include the step of adding 0.01-5.0 grams of Cab-O-Sil M5 SiO2 prior to the step of adding 54.99-50.0 grams of Gantrez MS955 to the mixture of glycerin and water. The Cab-O-Sil M5 SiO2 can be added to the mixture currently with the addition of water.

The hydrogels of examples 1-3 were provided to a panel of 13 denture wearers to compare with commercially available denture products Sea-Bond, Fixodent, and Super Poli Grip. The panel comprised men and women that compared these products over a period of 5 weeks and who were experienced using conventional denture pastes and powders. During the 1^{st} week the panel members were trained how to use the commercially available denture products in the same way and continued to utilize these products over the course of the 2^{nd} and 3^{rd} weeks. During the 4^{th} week the panel members were each provided with the hydrogels of examples 1-3 and asked to identify the preferred hydrogel. Example 1 was identified as the preferred hydrogel. During the 5^{th} week the panel members compared Sea-Bond, Fixodent and the preferred hydrogel (example 1). They provided comments with respect to their use of the hydrogel relative to Sea-Bond, Fixodent and any conventional pastes and powders that they use regularly. The panel members identified the hydrogel as having the beneficial qualities of providing an immediate hold and making the dentures feel natural and comfortable. The quickness of adhesive action for the hydrogel was identified as beneficial because known commercial denture adhesives take time to hold after initial application (ranging from approximately 5 to 30 minutes). The hydrogel was also identified as effectively conforming to the gums, lacking the problem of primary ooze associated with pastes and powders, and providing easy cleanup once the dentures are removed because the hydrogel mostly dissolved by the end of a day's use, resulting in a lack of residue. The benefits of the hydrogel of the present invention can be attributed at least in part to the fact that the adhesive characteristics of the hydrogel provided by the AVE/MA Salt component is already activated by the water retained in the hydrogel. This characteristic helps eliminate or minimize the problems associated with conventional pastes and powders, including primary ooze, a time delay until onset of adhesive activity, and presence of residual pastes and powders on the denture at the end of a day's use that have not fully hydrated resulting in a sticky residue that is difficult to clean.

The hydrogel of the present provides a beneficial denture adhesive with characteristics that are not realized by other proposed hydrogels, such as those disclosed in U.S. Pat. Nos. 6,583,225, 6,706,817, and 7,288,597. Unlike the hydrogels disclosed in these references, the glycerin utilized in the hydrogels of the above examples does not serve as a cross-linking agent for the AVE/MA Salt. To the extent AVE/MA Salt polymers are cross-linked, such cross-linking is achieved via the metal ion of the AVE/MA Salt. The hydrogel of the present invention utilizes glycerin to provide a moistening effect to the hydrogel, to suspend the AVE/MA Salt, and provide stability to the hydrogel over an extended period of time. The hydrogel also has a limited degree of swelling and resists dissolution in the presence of mouth moisture for an extended period time, while also retaining its improved adhesive characteristics. Further, the glycerin provides the therapeutic benefits of a warming sensation and lubricity in making the mouth feel better due to its emollient properties. These characteristics and the other beneficial features of the hydrogel of the present invention discussed herein are not achieved by known denture adhesive pastes and powders or other hydrogels for denture applications.

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A hydrogel denture adhesive, comprising:
an AVE/MA Salt in an amount of about 20 to about 80 percent by weight of the hydrogel;
a suitable humectant in an amount of about 1 to about 30 percent by weight of the hydrogel; and
water in an amount of about 20 to about 80 percent by weight of the hydrogel.

2. The hydrogel of claim 1, wherein the water is in an amount of about 30 to about 60 percent by weight of the hydrogel.

3. The hydrogel of claim 2, wherein the water is in an amount of about 35 to about 45 percent by weight of the hydrogel.

4. The hydrogel of claim 1, wherein the AVE/MA Salt is in an amount of about 30 to about 60 percent by weight of the hydrogel.

5. The hydrogel of claim 4, wherein the AVE/MA polymer salt is in an amount of about 45 to about 55 percent by weight of the hydrogel.

6. The hydrogel of claim 1, wherein the suitable humectant is in an amount of about 5 to about 20 percent by weight of the hydrogel.

7. The hydrogel of claim 6, wherein the suitable humectant is in an amount of about 8 to about 12 percent by weight of the hydrogel.

8. The hydrogel of claim 1, wherein the suitable humectant is selected from a group consisting of glycerin, polyhydric alcohols, ethylene glycol, propylene glycol, polyethylene glycol, sorbitol, and combinations thereof.

9. The hydrogel of claim 1, wherein the suitable humectant is glycerin.

10. The hydrogel of claim 1, further comprising an additional ingredient selected from a group consisting of an antimicrobial agent, an antibiotic, an anti-inflammatory agent, a dental desensitizing agent, an anesthetic agent, an anti-fungal, an aromatic, insulin, a steroid, an anti-neoplastic, a colorant, a preservative, a flavor component, a fragrance component, a sensation component, and combinations thereof.

11. The hydrogel of claim 1, further comprising a suitable rheological agent in an amount of about 0.1 to about 10 percent by weight of the hydrogel.

12. The hydrogel of claim 11, wherein the suitable rheological agent is in an amount of about 0.1 to about 5 percent by weight of the hydrogel.

13. A hydrogel denture adhesive prepared by a process comprising the steps of:
mixing a suitable humectant in an amount of about 1 to about 30 percent by weight of the hydrogel with water in an amount of about 20 to about 80 percent by weight of the hydrogel;
heating the mixture; and
dispersing an AVE/MA Salt in an amount of about 20 to about 80 percent by weight of the hydrogel in the mixture.

14. The hydrogel of claim 13, further comprising the step of heating the suitable humectant to 50-80° C. prior to the step of mixing the suitable humectant with water.

15. The hydrogel of claim 14, further comprising the step of cooling the dispersion or cooling the mixture prior to the step of dispersing the AVE/MA Salt.

16. The hydrogel of claim 13, further comprising the steps of mixing an AVE/MA Copolymer with a cross linking agent and a neutralizing agent, heating the mixture in solution to a cross-linking temperature and forming the AVE/MA Salt, and cooling the AVE/MA Salt.

17. The hydrogel of claim 16, wherein the cross-linking agent is a multivalent cation and the neutralizing agent is a monovalent cation.

18. The hydrogel of claim 17, wherein the cross-linking agent and the neutralizing agent are both alkali metal cations.

19. The hydrogel of claim 16, wherein the cross-linking temperature is about 85° C. to about 110° C.

20. The hydrogel of claim 13, wherein the water is in an amount of about 30 to about 60 percent by weight of the hydrogel.

21. The hydrogel of claim 20, wherein the water is in an amount of about 35 to about 45 percent by weight of the hydrogel.

22. The hydrogel of claim 13, wherein the AVE/MA Salt is in an amount of about 30 to about 60 percent by weight of the hydrogel.

23. The hydrogel of claim 22, wherein the AVE/MA polymer salt is in an amount of about 45 to about 55 percent by weight of the hydrogel.

24. The hydrogel of claim 13, wherein the suitable humectant is in an amount of about 5 to about 20 percent by weight of the hydrogel.

25. The hydrogel of claim 24, wherein the suitable humectant is in an amount of about 8 to about 12 percent by weight of the hydrogel.

26. The hydrogel of claim 13, wherein the suitable humectant is selected from a group consisting of glycerin, polyhydric alcohols, ethylene glycol, propylene glycol, polyethylene glycol, sorbitol, and combinations thereof.

27. The hydrogel of claim 26, wherein the suitable humectant is glycerin.

28. The hydrogel of claim 13, further comprising the step of mixing an additional ingredient selected from a group consisting of an antimicrobial agent, an antibiotic, an anti-inflammatory agent, a dental desensitizing agent, an anesthetic agent, an anti-fungal, an aromatic, insulin, a steroid, an anti-neoplastic, a colorant, a preservative, a flavor component, a fragrance component, a sensation component, and combinations thereof.

29. The hydrogel of claim 13, further comprising a suitable rheological agent in an amount of about 0.1 to about 10 percent by weight of the hydrogel.

30. The hydrogel of claim 29, wherein the suitable rheological agent is in an amount of about 0.1 to about 5 percent by weight of the hydrogel.
